(19) United States
(12) Patent Application Publication
(10) Pub. No.: US 2024/0120078 A1
(43) Pub. Date: Apr. 11, 2024

(54) DIGITAL ADVANCE HEALTHCARE DIRECTIVE MANAGEMENT

(71) Applicant: Nneka Obiajulu Sederstrom, Prior Lake, MN (US)

(72) Inventor: Nneka Obiajulu Sederstrom, Prior Lake, MN (US)

(21) Appl. No.: 18/483,391

(22) Filed: Oct. 9, 2023

Related U.S. Application Data
(60) Provisional application No. 63/379,069, filed on Oct. 11, 2022.

(51) Int. Cl. G16H 40/20; H04L 9/32

(52) U.S. Cl. CPC G16H 40/20; H04L 9/3228; H04L 9/3247

(57) ABSTRACT

The present disclosure is directed to systems and methods for managing digital advance healthcare directives. In one aspect, a method for creating a digital token for a digital directive is disclosed. The method comprising receiving the digital directive and a public key from a patient computing device, storing the digital directive in a healthcare directive data store, generating a digital token linking the stored digital directive to the public key, and publishing the digital token to a blockchain, wherein the digital token, when combined with a cryptographic signature from a private key paired with the public key, allows access to the digital directive.

### CROSS-REFERENCE TO RELATED APPLICATIONS

This application is a non-provisional of U.S. Provisional Patent Application No. 63/379,069, filed Oct. 11, 2022. The disclosure of the priority application in its entirety is hereby incorporated by reference into the presence application.

### BACKGROUND

Advance healthcare directives are legal documents describing a patient's wishes for their healthcare treatment if the patient is not able to make or express their wishes. The document could also be referred to as a living will, personal directive, advance directive, medical directive, or advance decision. Many advance healthcare directives also include information about a proxy that is selected by the patient to help make decisions for their healthcare. Some modern advance healthcare directives are digital and accessible online. Typical advance healthcare directives include a series of basic questions regarding healthcare decisions to be answered by the patient.

Blockchain technology is used to track, manage, and verify a digital ledger. In some examples, blockchain technology is used to track ownership of fungible digital assets, for example, crypto-currencies. In other examples, blockchain technology is used to track rights in non-fungible digital assets. For example, Non-Fungible Tokens (NFTs) are typically attached to digital files and are published on a blockchain in order to track ownership and other rights to the digital files.

### SUMMARY

In general terms, the present disclosure is directed to systems and methods for managing digital advance healthcare directives. Various aspects are described in this disclosure, which include, but are not limited to, the following aspects.

In one aspect, a method for creating a digital token for a digital directive is disclosed. The method comprising receiving the digital directive and a public key from a patient computing device, storing the digital directive in a healthcare directive data store, generating a digital token linking the stored digital directive to the public key, and publishing the digital token to a blockchain, wherein the digital token, when combined with a cryptographic signature from a private key paired with the public key, allows access to the digital directive.

In another aspect a healthcare directive server is disclosed. The healthcare directive server comprising a communication interface configured to establish digital communication with a healthcare directive data store, a patient computing device, and a blockchain, at least one memory device, and at least one processing device. The at least one memory device comprising instructions that, when executed by the at least one processing device, causes the processing device to receive a digital directive and a public key from the patient computing device, store the digital directive in the healthcare directive data store, generate a digital token linking the stored digital directive to the public key, and publish the digital token to the blockchain, wherein the digital token, when combined with a cryptographic signature from a private key paired with the public key, allows access to the digital directive.

In another aspect a method for establishing a digital directive on a blockchain is disclosed. The method comprising generating a private key and public key pair, receiving inputs for the digital directive, uploading the digital directive to a healthcare directive server with the public key, wherein the healthcare directive server is configured to store the digital directive in a health care directive data store, generate a digital token linking the digital directive with the public key, and publish the digital token to a blockchain, and accessing the digital directive via the blockchain using the private key to verify the participant user.

In yet another aspect, a method for providing emergency access to a digital directive is disclosed. The method comprising receiving, at a first computing device, an indication that a patient user is in an emergency situation, generating and displaying, at the first computing device, a temporary private key for a read-only access token, scanning, by a second computing device, the temporary read-only access key, providing, by the second computing device, the temporary private key to the blockchain to access the digital directive, to receive read-only access to a digital directive associated with the patient user, and rendering, on the display of the second computing device, a read-only copy of the digital directive associated with the patient user.

### DESCRIPTION OF THE DRAWINGS

The following drawing figures, which form a part of this application, are illustrative of described technology and are not meant to limit the scope of the disclosure in any manner.

FIG. 1 illustrates an example schematic diagram of an example healthcare environment in which the principles of the present invention may be employed.

FIG. 2 illustrates an example schematic diagram of the healthcare directive server of FIG. 1, in accordance with some embodiments of this disclosure.

FIG. 3 illustrates an example flow chart of an example method for managing digital advance healthcare directives, in accordance with some embodiments of this disclosure.

FIG. 4 shows example physical components of an example computing device usable in the healthcare environment of FIG. 1, in accordance with some embodiments of this disclosure.

FIG. 5 illustrates an example schematic diagram of a digital healthcare directive environment in which the principles of the present invention may be employed.

FIG. 6 illustrates an example schematic diagram of the healthcare directive server of FIG. 5, in accordance with some embodiments of the present disclosure.

FIG. 7 illustrates an example method for establishing a digital directive on a blockchain, in accordance with some embodiments of the present disclosure.

FIG. 8 illustrates an example method for creating a digital token for a digital directive, in accordance with some embodiments of the present disclosure.

FIG. 9 illustrates an example method for a delegate user to receive read-only access to the patients digital directive, in accordance with some embodiments of the present disclosure.

FIG. 10 illustrates an example method for receiving emergency access to a digital directive, in accordance with some embodiments of the present disclosure.

### DETAILED DESCRIPTION

Various embodiments of the present disclosure will be described in detail with reference to the drawings, wherein like reference numerals represent like parts and assemblies throughout the several views. Additionally, any examples set forth in this specification are not intended to be limiting and merely set forth some of many possible embodiments.

Broadly, the present disclosure is directed to systems and methods for managing digital advance healthcare directives. Patients can complete advance directives online using a dynamic questionnaire that provides prompts tailored to the patient. The patient can consult with healthcare professionals to discuss various aspects of their advance directive. The completed digital advance directive is easily accessed by the patient, medical professionals, and other interested parties.

The systems and methods disclosed for managing a digital advance healthcare directive provide several advantages. First, the digital advance healthcare directive is integrated to interface with a plurality of vendors (with different software platforms). This ensures that an up-to-date directive is provided in emergency situations. For example, in some emergency situations the patient may not be in a state to provide their wishes to a healthcare provider. The system and methods disclosed allow the provider to access an accurate directive using one or more vendors depending on what systems are available at the time of an emergency.

Additionally, life events (e.g. marriage, birth of a child, or change in medical circumstances) may change the patient's wishes for their directive. The systems and methods disclosed provide different ways automatically notify a patient to review their directive. In some examples this notification is done automatically at periodic times to ensure the directive is up-to-date. In other examples, this notification is sent to a patient automatically based on a scheduled procedure. The systems and methods manage a patient directive to ensure that the various vendors (using different technology platforms) are also updated to include the current directive for a patient.

The methods and systems disclosed further include integrations with one or more electronic medical record (EMR) systems to automate the input of standard patient information so that a patient does not need to enter the entirety of their information when completing their directive.

Next, the methods and systems disclosed herein provide a dynamic directive based on the patient. The dynamic directive improves the accuracy of the directive by selecting prompts based on the patient information. The methods and systems disclosed also allow a patient to communicate with an expert in real time while completing the directive to improve the patients understanding and comfort with the directive.

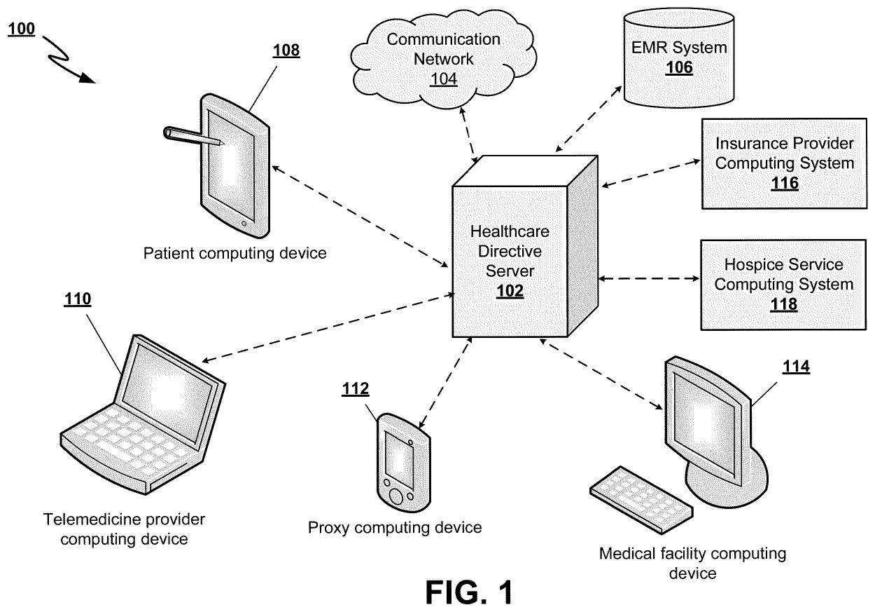

FIG. 1 is a schematic diagram of an example healthcare environment 100. The healthcare environment 100 operates to care for patients, including guiding the patients through preparing an advance healthcare directive. The healthcare environment 100 includes a healthcare directive server 102, a communication network 104, an electronic medical record system 106, a patient computing device 108, a telemedicine provider computing device 110, a proxy computing device 112, a medical facility computing device 114, an insurance provider computing system 116, and a hospice service computing system 118. Each component of this healthcare environment 100 communicates with each other via wired or wireless connections. In some embodiments, additional components can communicate with the healthcare environment 100 through the communication network 104. In some embodiments, some of the components shown in FIG. 1 are optional.

The healthcare directive server 102 operates to manage digital advance healthcare directives for a plurality of patients. Questionnaires are presented to the patients on the patient computing device 108. It will be appreciated that while a single patient computing device 108 is illustrated in FIG. 1, there are actually a plurality of devices 108 which may be used. More specifically, each patient reviewing and/or filling out the questionnaires may use one or more of their own devices 108 or may use a shared device 108.

Similarly, some embodiments include multiple healthcare directive servers 102. In these embodiments each of the multiple healthcare directive servers may be similar and provide similar functionality. Alternatively, in these embodiments, the multiple healthcare directive servers may perform specialized functions or specialized services. In some examples, the multiple healthcare directive servers may use a cloud computing technology to provide greater capacity, redundancy, or provide services from multiple geographic locations.

Additionally, a person of ordinary skill in the art will recognize that privacy and security is critical to the healthcare directive server 102. Accordingly, privacy and security may preclude certain server architecture solutions or may require a specific implementation. Additional services may also be required to ensure the privacy and security of the healthcare directive server 102.

Responses to the questionnaires are recorded in the electronic medical record (EMR) EMR system 106. Patients can consult with medical professionals regarding questions about the healthcare directive. If the medical professionals are communicating with the patients remotely, they may operate a telemedicine provider computing device 110. After the digital advance healthcare directive is completed for a patient, a proxy can be notified on the proxy computing device 112. Medical professionals can access the patient's healthcare directive using the medical facility computing device 114 to inform decision-making regarding the patient's medical care. The patient's insurance provider can be notified that the patient has completed a healthcare directive via the insurance provider computing system 116. Additionally, the healthcare directive server 102 can communicate with a hospice service computing system 118 to provide patients with access to hospice resources in conjunction with completing digital advance directives.

One or more components of the healthcare environment 100 are in communication with each other via a communication network 104. The communication network 104 may include any type of wireless network, a wired network, or any communication network known in the art. For example, wireless connections can include cellular network connections and connections made using protocols such as 802.11a, 802.11g, 802.11n, and/or 802.11ac.

In some embodiments, one or more components of the healthcare environment 100 are integrated with a health information exchange. The health information exchange is configured to electronically move health data (e.g., clinical data) to different components of the healthcare environment 100. In some embodiments, the healthcare information exchange uses a separate connection from the communication network 104.

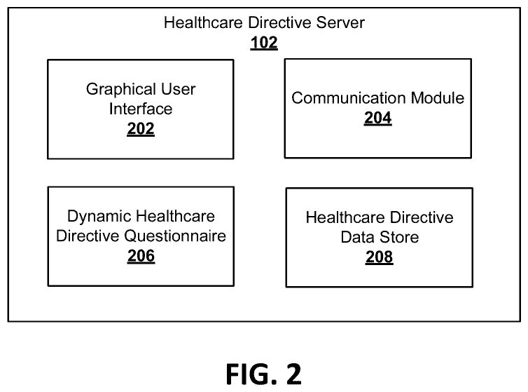

FIG. 2 is a more detailed schematic diagram of the healthcare directive server 102 of FIG. 1. The healthcare directive server 102 manages healthcare directive questionnaires, patient's completed directives, and communication of the directives to the relevant parties. The healthcare directive server 102 includes at least one processing device and at least one memory device (not shown). The memory device includes instructions to operate a graphical user interface 202, a communication module 204, and a dynamic healthcare directive questionnaire 206 when executed by the processing device. The memory device also includes a healthcare directive data store 208.

The graphical user interface (GUI) 202 operates to present a visual display for computing device users to access, enter, edit, and delete healthcare directive information. In some embodiments, the GUI 202 presents a dynamic healthcare directive questionnaire to a patient or to an individual assisting a patient in preparing a healthcare directive. In some embodiments, the individual is a medical professional such as a physician, nurse, ethicist, or designee. In some embodiments, the individual is a proxy or a loved one. Responses to the questionnaire are received at the GUI 202 through inputs provided at a computing device.

In some embodiments, the GUI 202 receives input from a computing device requesting to view a completed healthcare directive. In some embodiments, the computing device is a telemedicine provider computing device 110, a proxy computing device 112, or a medical facility computing device 114 accessing a patient's healthcare directive in order to counsel the patient or to aid in making decisions for the patient's healthcare. The patient's digital healthcare directive is then displayed on the GUI 202.

In some embodiments, a patient may utilize the GUI 202 to edit or delete their digital healthcare directive. For example, the GUI 202 receives input from a patient computing device 108 of the patient editing or deleting their digital healthcare directive. In other examples, the inputs are received by a proxy computing device 112 where the inputs are submitted by a proxy for the patient.

The communication module 204 operates to manage communication of healthcare directive information to and from the healthcare directive server 102. In some embodiments, the communication module 204 communicates with patient computing devices 108, telemedicine provider computing devices 110, proxy computing devices 112, and medical facility computing devices 114. In some embodiments, dynamic healthcare directive questionnaires 206 are communicated to patient computing devices 108 and telemedicine provider computing devices 110 and completed healthcare directives are received in return. In some embodiments, the communication module 204 manages requests for information received from EMR systems 106, insurance provider computing systems 116, and medical facility computing devices 114. In some embodiments, the communication module 204 directs communication of patient healthcare directives to the requesting computing system for display on a screen of a computing device. In some embodiments, the communication module 204 coordinates communication between patients and medical professionals regarding appointments, questions about healthcare directives, information about a patient's health, and information in a patient's healthcare directive.

The dynamic healthcare directive questionnaire 206 operates to present to a patient (or individual aiding a patient) a series of prompts or questions regarding various aspects of the patient's healthcare wishes. Further details regarding an example dynamic questionnaire are provided below.

The healthcare directive data store 208 operates to store information regarding patients' healthcare directives. In some embodiments, a record of the patients that have completed a digital healthcare directive is kept. In some embodiments, partially completed digital healthcare directives are stored and a patient can later access their digital healthcare directive to complete it. In some embodiments, completed digital healthcare directives are stored for later access by medical facilities, proxies, etc.

Preferably the stored healthcare directives are securely stored in the healthcare directive data store 208 and may be accessed in whole or in part only by authorized personnel (described in more detail below). Accordingly, devices and systems which secure the data in the healthcare directive data store 208 are preferably provided. For example, password protection, multi-factor authentication, and block chain type systems may be used among others.

Although only one healthcare directive data store 208 is illustrated in FIG. 2, some examples include multiple healthcare directive data stores 208. In these examples, the multiple healthcare directive data stores 208 can be configured to provide a modular, scalable, and redundant storage. In some examples, a cloud computing technology is used.

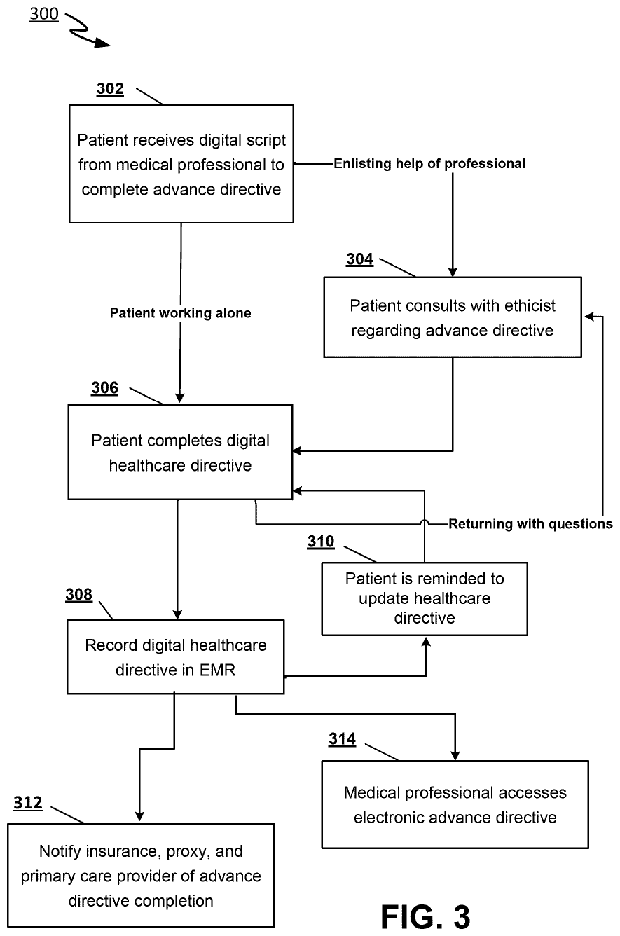

FIG. 3 is an example flowchart of a method 300 for managing digital advance healthcare directives. In some embodiments, this method 300 is performed by one or more components in the healthcare environment 100 of FIG. 1. In particular, the healthcare directive server 102 performs many aspects of the method 300.

At operation 302, the patient receives a digital script from a medical professional to complete an advance directive. In some embodiments, a patient's need for an advance directive is discussed with a medical professional during an appointment. The appointment could be in-person or online (telemedicine). In some embodiments, the medical professional can add a note to a patient's EMR indicating that the patient needs to complete an advance directive and include a link to an online digital healthcare directive. In some embodiments, a patient that does not already have an advance directive recorded in their EMR may be asked when they check-in for an appointment or make an appointment if they would like to speak to a medical professional about completing a healthcare directive.

In some embodiments, the medical professional could write a digital script for the patient to visit an ethicist or designee to discuss their advance directive. The patient could then be assisted in making an appointment with the ethicist or designee through online scheduling or the aid of an administrative professional at the healthcare facility the patient is visiting. If the patient enlists the help of a professional to aid in completing the advance directive, the method proceeds to operation 304.

In some embodiments, a patient accesses a digital advance directive questionnaire without being provided with a script or prompt from a healthcare professional. In some embodiments, the patient can download a software application or access a web service to complete a directive.

At operation 304, the patient consults with an ethicist or designee (or other medical professional) regarding their advance directive. The consultation could be done in person or online (telemedicine). In some embodiments, the consultation is performed through a digital communication tool allowing for face-to-face conversation between the ethicist or designee and the patient (or patient's proxy). In some embodiments, the patient could simply have a phone conversation with an ethicist or designee regarding questions that the patient has regarding their health directive. In some embodiments, a patient's physician can consult directly with the ethicist or designee to answer a patient's questions or to guide a patient in completing a directive.

After this consultation, the method proceeds to operation 306. However, in some embodiments, operation 306 occurs at the same time as operation 304. In some embodiments, the ethicist or designee assists in the completion of the healthcare directive based on discussions with the patient about their healthcare wishes.

If the patient is working alone on their advance directive, the method proceeds to operation 306. Note that the advance directive could be completed by the patient (if the patient is an adult), a parent or guardian of the patient (if the patient is under 18 years of age), or a proxy individual appointed by the patient or designated legal guardian to assist with completing the questionnaire. At operation 306, the patient completes a digital healthcare directive. In some embodiments, the patient utilizes a patient computing device such as a smartphone, tablet, laptop, or desktop computer to complete a dynamic questionnaire. Details of the questionnaire are provided below.

In some embodiments, if the patient has questions while completing their directive, they can make an appointment to consult with an ethicist or designee or other professional and the method returns to operation 304.

At operation 308, the patient's digital healthcare directive is recorded in the patient's electronic medical record (EMR). In some embodiments, the patient uploads a completed digital healthcare directive to their EMR through a patient portal online. In some embodiments, if the patient has logged into a patient portal linked to their EMR, the digital healthcare directive will be automatically saved with the patient's EMR.

In some examples, the data may be anonymized and collected to train a model. In these examples, before collecting and anonymizing the data the patient is asked whether they consent to having their anonymized data used to train a model. The model is trained to analyze and make insights on the dynamic directive. In these examples, the model is used to further enhance the dynamic directive. For example, the model may select certain prompts based a patient profile or specific follow up prompts. A model may also be trained to recommend the patient receive a second opinion based on the received responses to directive questions. In some examples, the model is trained using machine-learning, artificial intelligence, or business intelligence.

Periodically, the method proceeds to operation 310 and the patient is reminded to update their healthcare directive. In some embodiments, reminders are communicated to the patient electronically. For example, the patient could receive emails triggered by their EMR indicating that the patient should review their healthcare directive and potentially update the directive. In some embodiments, the patient's EMR could be flagged to indicate to a medical professional to discuss updating the patient's healthcare directive at the patient's next appointment. In some embodiments, the frequency of the reminders is determined based on the patient's age.

In some embodiments, after the patient's healthcare directive is completed, one or more parties can be notified electronically. At operation 312, one or more of an insurance, proxy, and/or primary care provider are notified of advance directive completion.

In some embodiments, after a proxy is notified that they have been selected by a patient, the proxy is presented with multiple options. One option presents the proxy with information to contact the patient's ethicist, designee and/or medical professional to address any questions or concerns that the proxy has. Another option allows the proxy to decline their appointment as the patient's proxy. This could be because the person named to be the patient's proxy does not feel that they can be a good decision maker or because they do not want the responsibility. The option could present an “opt out” button for the proxy to decline being the named decision maker, which triggers a notification to a patient to identify an alternative. In some embodiments, the patient has appointed a primary proxy decision maker and a back-up proxy decision maker. In such embodiments, if the primary proxy opts out, the back-up proxy is automatically notified. The back-up proxy can also be presented with an option to opt-out.

At operation 314, one or more medical professionals can access the patient's electronic advance directive. In some embodiments, it is accessed from the patient's EMR. In some embodiments, the directive could be stored on a personal computing device of the patient such as a smartphone. In some embodiments, the directive is stored in a digital database of directives managed by an organization such as a state-sponsored organization.

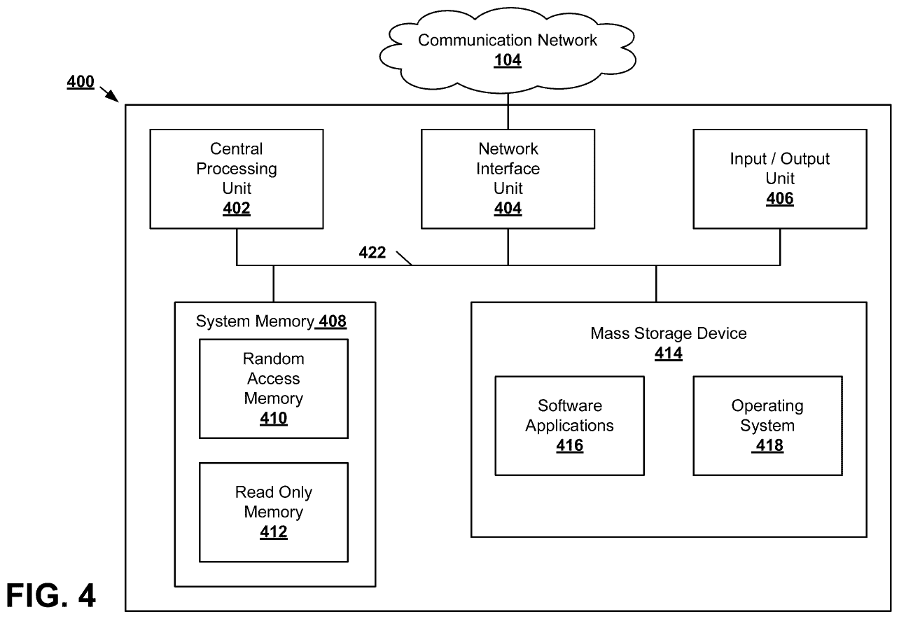

FIG. 4 is a block diagram illustrating an example of the physical components of a computing device 400. The computing device 400 could be implemented in various aspects of the healthcare environment 100, such as the patient computing device 108, telemedicine provider computing device 110, and proxy computing device 112. Components of the computing device 400 can also be incorporated into other devices and systems described herein, such as the insurance provider computing system 116 and EMR system 106.

In the example shown in FIG. 4, the computing device 400 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 400, such as during startup, is stored in the ROM 412. The computing device 400 further includes a mass storage device 414. The mass storage device 414 is able to store software instructions 416 and data.

The mass storage device 414 is connected to the CPU 402 through a mass storage controller (not shown) connected to the system bus 422. The mass storage device 414 and its associated computer-readable storage media provide non-volatile, non-transitory data storage for the computing device 400. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain embodiments, the computer-readable storage media comprises entirely non-transitory media.

Computer-readable storage media includes volatile and non-volatile, removable and non-removable media implemented in any method or technology for storage of information such as computer-readable software instructions 416, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 400.

According to various embodiments, the computing device 400 can operate in a networked environment using logical connections to remote network devices through a communication network 104, such as a wireless network, the Internet, or another type of network. The computing device 400 may connect to the communication network 104 through a network interface unit 404 connected to the system bus 422. It should be appreciated that the network interface unit 404 may also be utilized to connect to other types of networks and remote computing systems. The computing device 400 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 400 can store software instructions 416 and data. The software instructions include an operating system 418 suitable for controlling the operation of the computing device 400. The mass storage device 414 and/or the RAM 410 also store software instructions 416, that when executed by the CPU 402, cause the computing device 400 to provide the functionality discussed in this document.

### Example System Setup

The following provides information for one possible setup of a digital directive system. Other embodiments and implementations are possible.

Digital Directive Setup

As discussed above, in many embodiments the dynamic directive provides seamless communication between patient, proxy, provider, payer, ethicist or designee. Additionally, the dynamic directive is often configured to interface with an EMR and/or a hospice connection. Each of the various users and/or systems must maintain certain data protection requirements. In some examples, the users must agree to upholding confidentiality before using the digital directive. Additionally, the users can be required to agree to upholding confidentiality rules before entering the app or setting up an account.

Different users may have different levels of access. For example, a patient and a provider may have complete access to the dynamic directive. In contrast a proxy does not have access to edit or change the dynamic directive. For example, the proxy may only have access to provide the directive to a healthcare provider. In some examples, the proxy is prompted to agree to serve as a decision maker for a patient. If the primary proxy declines to serve as a decision maker, then a secondary proxy is notified. In some examples, a payer only has access related to whether the patient has completed the dynamic directive or if the patient makes any revisions or updates to the dynamic directive. In some examples, an ethicist or designee has access to the dynamic directive in a read only format. Accordingly, the ethicist or designee only has access necessary to provide support and clarification for a patient and/or a proxy.

System Integration Needs

In some embodiments dynamic directive is integrated with systems from multiple vendors. Accordingly, the dynamic directive may include an application programming interface (API) layer to facilitate these integrations. For example, the application may have an API layer which allows the various vendors to request different features which the application may provide a response to properly authenticated parties. Similarly, the application may interface with other vendor's API's. For example, the application may interface with an EMR system to store the completed directive.

In some embodiments the dynamic directive is integrated with one or more electronic medical record (EMR) systems. For example, the dynamic directive is uploaded to an EMR system with a timestamp. Additionally, the dynamic directive is integrated with a patient portal. The patient portal may have a folder which gives the patient easy access to the dynamic directive. The patient portal may also send updates to the patient. For example, the patient portal may notify the patient to discuss the dynamic directive with the patient's primary care physician at a clinical visit.

In some embodiments, the dynamic directive is integrated with a standards based healthcare IT platform. In some embodiments, the standards based healthcare IT platform is configured to enable various computer applications to securely connect and run across a healthcare system. In some embodiments, the EMR system is integrated with the standards based healthcare IT platform. For example, the EMR system may store the medical data in a manner which supports the standard defined by the healthcare IT platform. In some embodiments, the healthcare IT platform may define data security standards, format standards, as well as other standards for the dynamic directive.

In some examples, the patient portal may prompt a patient to questions related to sharing or discussing the dynamic directive with a new or existing specialist. For example, the patient may be asked to share the dynamic directive through the patient portal with other clinicians. In some examples a pop-up notice is displayed for the patient for when a new appointment is made with a new clinician to see if the patient is interested in sharing the dynamic directive with the new clinician.

In some examples, the EMR system for the primary care physician may be different from the EMR system used by a specialist. The dynamic directive may be configured to interface with a plurality of different EMR systems. In some examples, some sections of the dynamic directive are automatically populated with information saved in the EMR system. For example, the EMR system can populate the personal data section for a patient.

In some embodiments the primary physician clinic sends reminders through the EMR system and/or the patient portal to remind the patient to review the dynamic directive at or before a clinic visit. These reminders may be automated to be sent out at a predetermined time. For example, the reminders to review the dynamic directive may be sent every 6 months or every year. In some examples, a record in the EMR system is updated when the patient updates the dynamic directive and the primary physician clinic is notified about the update.

The system is also be configured to send additional notifications to specific users. These notifications, in some examples, are sent over email or text message. Other secure messaging applications can also be used. In one example the system may send a secure email notification to a proxy based on different events. For example, the message is sent to the proxy to prompt the proxy to save the dynamic directive on the proxy's personal account under the patient's name. In some examples, the proxy has a professional account and a personal account and may receive notifications from both accounts. In another example the proxy receives notifications of any updates or changes to the patient's dynamic directive.

Similarly, the ethicist or designee may receive notifications from the system. For example, the ethicist or designee may receive a message with the patient's dynamic directive before meeting with the patient. In some examples, this message is removed after the ethicist or designee meets with the patient. The ethicist or designee may also receive notifications when the patient makes updates or changes to their dynamic directive.

The ethicist (or designee) and proxy may have separate accounts with their own dynamic directives. This allows these users to keep their personal and professional accounts separate. In some examples, the professional and personal accounts are accessed from the same part of the application. For example, a user-interface may include one selection for “Digital Directives” which presents a display with a “My Directive” selection linking to a personal directive and a “Proxy” selection linking to a list of directives the individual is proxy to. Each selection may include selections for accessing related personal or proxy services. Alternatively, professionals can utilize a separate software application. For example, a professional application and a personal application.

In some examples, the application prompts the patient or proxy to get a second opinion after meeting with an ethicist or designee. The application may select a physician or other expert for the second opinion based on the patient's demographic information. A patient may wish to get a second opinion from a physician with a similar demographic background. For example, a patient may receive a request asking if the patient wishes to speak to a physician of the same race to discuss the dynamic directive. Providing a second opinion in this manner may improve health equity by enhancing the accuracy of the dynamic directive and improving clinical outcomes for the patient.

In some examples, the application may present scenarios to the user which allows a user to understand how the dynamic directive would be used in different scenarios. The patient may then update the dynamic directive based on the second opinion or on the presented scenarios.

In some examples, the application may generate a machine-readable code which when scanned by a device transfers the patient ID associated with the dynamic directive to an approved user. In some examples the machine-readable code is a QR code. In some examples, when the code is scanned by a properly authenticated device a message is displayed on the device informing the healthcare provider that the patient has a directive.

In some examples, the mobile computing device may detect that the patient is in a critical condition, using sensors on a phone or connected smart device like a smart watch. Any other sensor detecting wearable devices can be configured to operate individually or with the mobile computing device to detect whether the patient is in critical condition. After detecting that the patient is in critical condition the phone may automatically display a patient ID number or a QR code associated with the patient ID. Similarly, the mobile application may automatically ping the EMR system to notify the healthcare provider that the patient has a dynamic directive.

In other examples, the application may interface with an emergency contact feature in a mobile computing device. For example, some mobile computing devices include a feature to contact an emergency contact without unlocking the phone. The digital directive application may interface which such a feature to allow a user to contact a directive contact (e.g. a proxy, or designee) without unlocking the phone when the device owner is experiencing a medical emergency.

In other examples, a care provider may have a QR code which facilitates the sign-up of a new patient to use the dynamic directive. In this manner a doctor may instruct the patient to complete the questionnaire after being directed to application.

### Questionnaire

The following outlines a series of prompts that are presented to a patient in a dynamic questionnaire for completing a dynamic advance directive. Note that the questionnaire could be completed by the patient (if the patient is an adult), a parent or guardian of the patient (if the patient is under 18 years of age), a medical professional caring for the patient, or a proxy individual appointed by the patient to assist with completing the questionnaire. This is just one example of the information that a patient might be requested to provide. Other embodiments and implementations are possible.

In some examples, the prompts provided to the patient are based on the patient ID or an account associated with the patient. Similarly, the prompts presented may be based on the patient's medical history or demographic information.

Introduction Statement

The Digital Directive is a chance for you to take control of your right to decide about your care based on your values. It is designed to help your family, friends, and providers know what is important to you when you are not able to speak for yourself. You have the option to revisit, revise, and cancel this directive at any time. Your voice is what matters.

Personal Data
Demographic data
   i. Name
   ii. Address
   iii. Married/Partner
   iv. Gender Identity
   v. Race/Ethnicity
   vi. Optional: Religion
   vii. DOB
   viii. Primary doc info
   ix. Subspecialist physician info
DPA/medical decision maker
   i. Name and contact info
   ii. Relationship to account owner
   iii. Are they aware of being the DPA
Values and quality of life
   i. List of values that you hold dear (click all)
      1. Engaging with family and/or friends
      2. Being active
      3. Independence
      4. Truthfulness
      5. Frank and Direct communication
      6. Ability to work
      7. Travel and leisure activities
      8. Engaging in pastimes and/or hobbies (Free text to list top 3)
   ii. Would you want your quality of life to be a factor in decision making and if so, what values do you hold as the highest
      1. Ability to communicate effectively
      2. Ability to walk or be mobile without assistants or with assistance
      3. Recognizing and engaging with family and/or friends in
      meaningful ways
      4. Ability to see, hear, touch and/or feel
      5. Ability to care for my personal body needs
      6. Be able to stay in my home for as long as I can
   iii. Things about me I want my caregivers to know:
      1. Free text about hobbies and other things they may want caregivers to know
      2. Opportunity to be specific on values and quality of life indicators
   iv. I want my doctors to only provide the medically appropriate options to achieve my values and goals to my DPA. (filling out directive is optional if this is checked)

My Advance Directive and Wishes
If I am in a permanently vegetative state (coma), these are my wishes (to include starting and/or stopping of interventions):
   i. Vent yes/no
   ii. Feeding tube yes/no
   iii. Dialysis y/n
   iv. ECMO/LVAD/adv heart devices yes/no
   v. Comfort care medication/pain meds yes/no
   vi. Autopsy yes/no
   vii. Accept time limited trial of up to 2 weeks on any of these features yes/no
   viii. Other specific requests
   ix. Hospice referral
If I have a terminal or end stage disease or condition defined as having less than 6 months of life left, these are my wishes (to include starting and/or stopping of interventions):
   i. Vent yes/no
   ii. Feeding tube yes/no
   iii. Dialysis y/n
   iv. ECMO/LVAD/adv heart devices yes/no
   v. Comfort care medication/pain meds yes/no
   vi. Autopsy yes/no
   vii. Accept time limited trial of up to 2 weeks on any of these features yes/no
   viii. Hospice referral
I am willing to engage in any aggressive interventions for a limited time trial. Continuation of these interventions must be based on clear medical evidence of my significant clinical improvement. If there are no evidence to justify continuing, I wish to have these interventions stopped. Yes/No
Other specific requests

Religious Values
Check boxes for religious group with free text option to describe wishes

Witnessed and signed through electronic means/e-sign/notification to DPA

I would like to speak with a health professional about my advance directive
In person or telemedicine visit with Primary care doc
Telemedicine visit with an Ethicist or designee

After completing the dynamic directive, an email can be sent to the patient's primary care provider, any relevant specialists, DPA, and the patient's EMR. Following completion, reminders are sent to the patient on a scheduled basis. For individuals under 65 years of age, yearly notifications are sent requesting for the patient to renew or reaffirm their directive. For individuals 65 and over, the notifications are sent every 6 months.

Digital Directive with Blockchain Integration

In some embodiments, a digital directive is integrated with blockchain technology. In some embodiments, the digital directive is generated using the methods and systems described above. Blockchain technology is used to provide secure decentralized access to the digital directive to authorized users. In some embodiments, a digital token (e.g., a non-fungible token “NFT”) is recorded on a blockchain. The digital token includes a link to the digital directive stored at a digital directive data store. Users are provided private keys which allow users to access, with authorization, digitals directive using the digital tokens.

In some embodiments, different keys are provided to different users. For example, a patient user (e.g., the user that is the subject of the digital directive) uses a private key which allows the patient user to access and edit the digital directive and a delegate user (e.g., a family member of the patient) uses a private key which provides read-only access. Another key can be transferred to a user in emergency situations to provide read-only access. For example, when a patient is in an emergency state and unable to provide the digital directive information.

In some embodiments, the blockchain technology allows various authorized users to access a digital directive. For example, multiple different healthcare providers, medical record providers, family members, medical professionals, can access digital directives using different applications to interface with the blockchain. Private or public blockchains can be used in different embodiments.

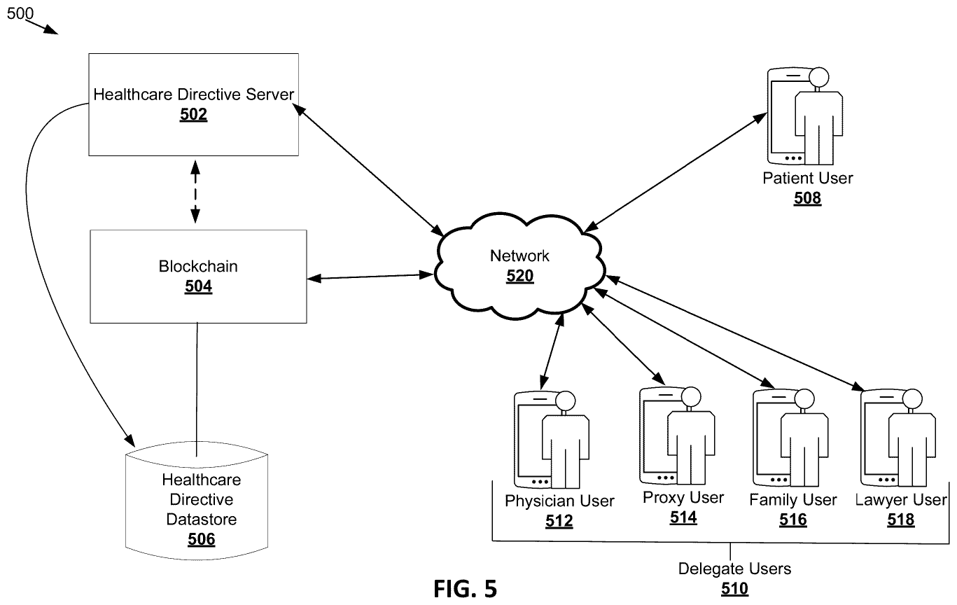

FIG. 5 illustrates an example schematic diagram of a digital healthcare directive environment 500 in which the principles of the present invention may be employed. The digital healthcare directive environment 500 includes a healthcare directive server 502, a blockchain 504, a healthcare directive data store 506, a patient user 508, delegate users 510, and a network 520.

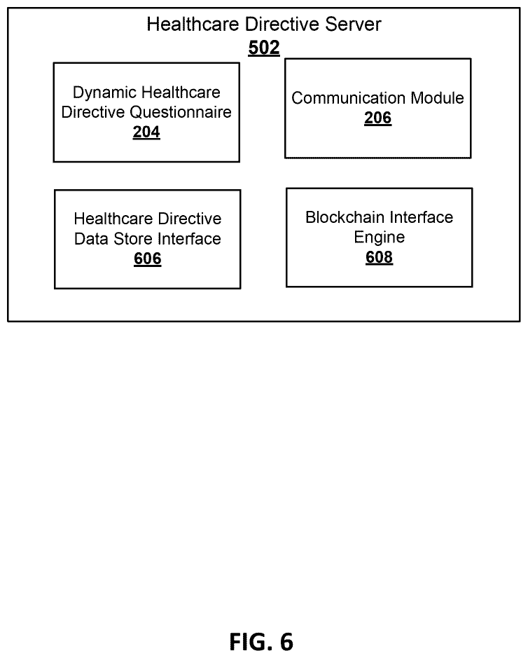

The healthcare directive server 502 manages digital directives for a plurality of patient users including the patient user 508. In the embodiment shown, the healthcare directive server 502 is integrated with the blockchain 504. In some embodiments, the healthcare directive server 502 operates with a computing device for the patient user 508 to prompt a patient user to complete a digital directive. In some embodiments, the healthcare directive server 502 receives a completed directive from a patient user with a public key of the patient user, stores the completed digital directive in the healthcare directive data store 506, generates a digital token linking the public key with the stored digital directive, and publishes the digital token to the blockchain 504. The healthcare directive server 502 is another example of the healthcare directive server 102 illustrated and described in reference to FIGS. 1 and 2. Another example of the healthcare directive server 502 is illustrated and described in reference to FIG. 6.

The blockchain 504 stores a digital ledger which tracks access rights for digital directives stored in the healthcare directive data store 506. In some embodiments, the blockchain 504 is a public blockchain. In some embodiments, the blockchain 504 is a private blockchain. In some embodiments, the blockchain 504 comprises a plurality of computers linked over the network 120 to record and verify a common digital ledger. In some embodiments, the plurality of computers are linked in a peer-to peer network. Various platforms for blockchains can be used.

In some embodiments, the blockchain 504 is accessible by a plurality of different applications including different healthcare provider applications, and/or medical record applications. Additionally, the blockchain is accessible by applications running on a computing device of the patient user 508 (sometimes referred to as patient user computing device) and on computing devices of the delegate users 510 (sometimes referred to as delegate user computing device).

The healthcare directive data store 506 store digital directives for a plurality of users. In some embodiments, the healthcare directive data store 506 is stored at a separate computing system form the healthcare directive server 502. In other embodiments, the healthcare directive data store 506 is integrated with the healthcare directive server 502. For example, as shown in FIG. 2 with the healthcare directive data store 208 in the healthcare directive server 102. In some embodiments, the healthcare directive data store 506 is stored in a decentralized database.

The patient user 508 is a user who is the subject of the digital directive. In some embodiments, the patient user 508 completes the digital directive with an ethicist. Examples for completing the digital directive are described above. In some embodiments, ethicist user also receives read and write access to some or all portions of the digital directive.

Examples of delegate users 510 include a physician user 512, a proxy user 514, a family user 516, and a lawyer user 518. Other examples include provider accounts, hospital accounts, emergency medical records (EMR) accounts, payor accounts, friend accounts, hospice accounts, nursing home accounts, and or accounts/users for other decision makers. In some embodiments multiple keys for delegate users must be submitted with the digital token stored on the blockchain in order to access a read-only copy of the digital directive. For example, a family user 516 may be required to provide their private key to a provider which has a second private key, where the combination of private keys are required to access the digital directive for the patient user. In some embodiments, a patient user can provide different levels of access to different delegate users. For example, a friend account may have access to only some of the information included in the digital directive but a family account could have read-only access to all of the information in the digital directive.

The network 520 operates to connect various computing systems including computing systems for the patient user, delegate users, the health care directive server, computing devices of the blockchain 504, etc. In some embodiments, the network 520 is a public network, such as the Internet.

FIG. 6 illustrates an example schematic diagram of the healthcare directive server 502 of FIG. 5, in accordance with some embodiments of the present disclosure. The healthcare directive server 502 includes a dynamic healthcare directive questionnaire 204, a communication module 204, a healthcare directive data store interface 606, and a blockchain interface engine 608.

The dynamic healthcare directive questionnaire 206 and communication module 204 operate similar to the healthcare directive questionnaire 206 and communication module 204 illustrated and described in reference to FIG. 2.

The healthcare directive data store interface 606 interfaces with a healthcare directive data store to store and update digital directives for a plurality of patient users. In some embodiments, the healthcare directive data store is integrated with the healthcare directive server 502. The healthcare directive data store interface 606 interfaces to the healthcare directive data store to send completed/updated directives to the datastore. In some embodiments, after the digital token is published to the blockchain, authorized users can access the digital directive directly via the blockchain.

The blockchain interface engine 608 operates to generate digital tokens attached to digital directives and publish the generated tokens to the blockchain. The tokens are generated to confirm the identity of a user via a private key securely stored on a user's device. For example, the user can send a request to access a digital directive with a cryptographic signature generated with the private key such that the blockchain can verify that the user is allowed to access the corresponding digital directive. In other embodiments, the user device (e.g., patient user device, or the delegate user device) directly interfaces with the blockchain to generate and publish the token. In some of these embodiments the healthcare directive server 502 is not included and the functions described herein are performed on the user's computing device and the blockchain.

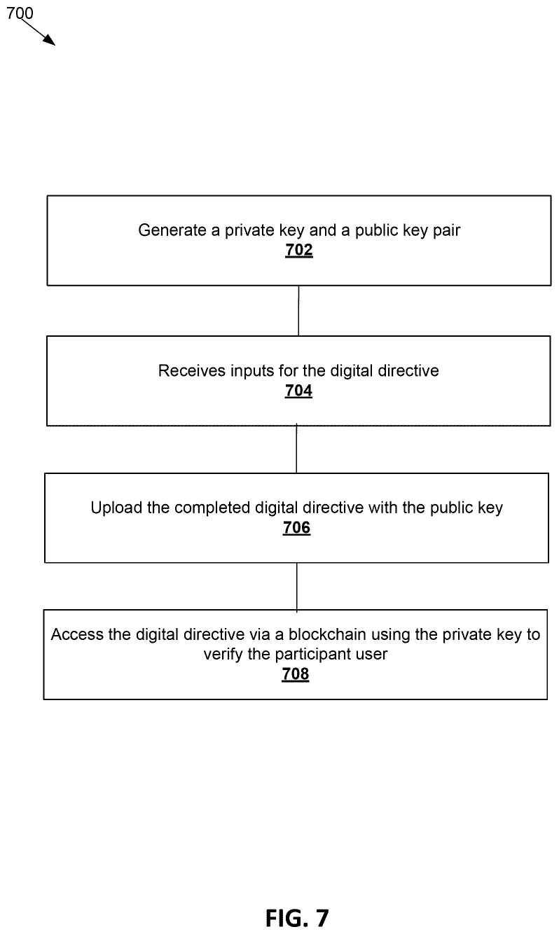

FIG. 7 illustrates an example method 700 for establishing a digital directive on a blockchain, in accordance with some embodiments of the present disclosure. The method 700 is performed at a patient's computing device, for example, by the patient user 508 as shown in FIG. 5. The method 700 includes the operations 702, 704, 706, and 708.

The operation 702 generates a private key and public key pair. In some embodiments, the public key is an address for a digital wallet. In some embodiments, the private key is used to generate a cryptographic signature which verifies the participant user.

The operation 704 receives inputs to complete the digital directive. In some embodiments, the digital directive is the dynamic digital directive illustrated and described herein. In some embodiments, a patient user completes the digital directive with an ethicist.

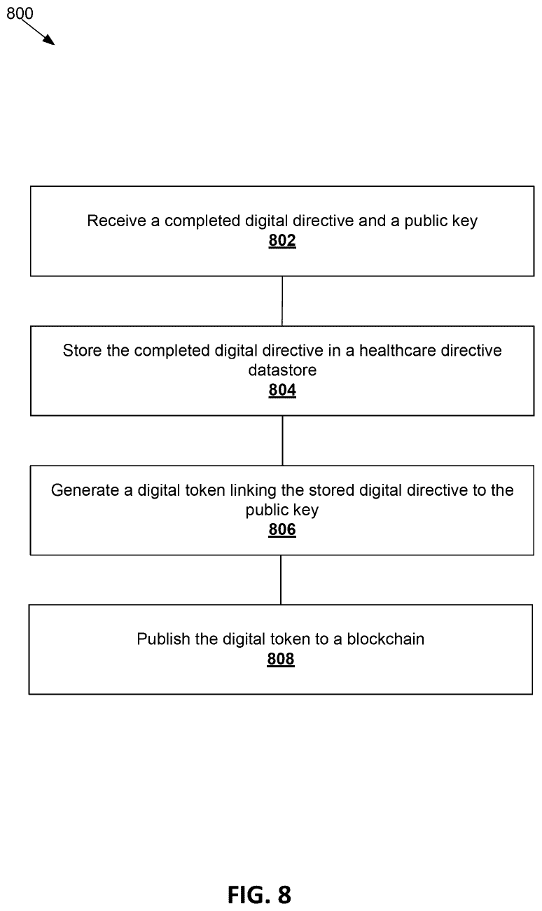

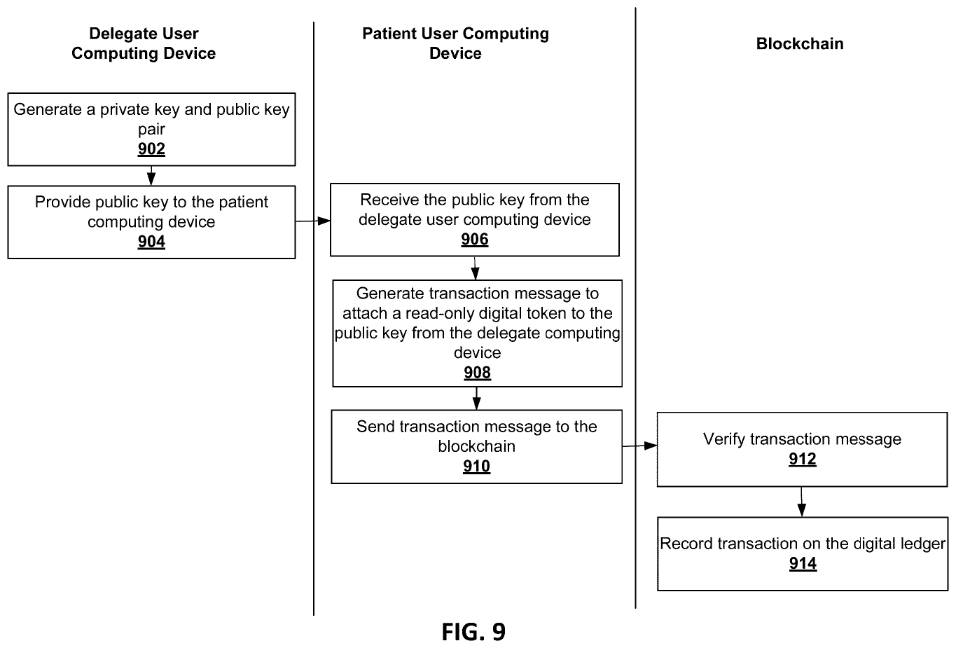

The operation 706 uploads the completed digital directive to a healthcare directive server with the public key of the participant user. In some embodiments, the digital healthcare directive server stores the digital directive in a digital directive datastore and generates a digital token linking the stored digital directive with the public key, such that the participant user can access and edit the digital directive. An example method for generating a digital token is illustrated and described in reference to FIG. 8. In some embodiments, the digital token also allows a participant user (with the private key) to transfer read-only access to the digital directive. An example method for providing read-only access to a delegate user is illustrated and described in reference to FIG. 9.

The operation 708 accesses or edits the digital directive via a blockchain using the private key to verify the participant user. In some embodiments, a patient user sends an access request to the blockchain with a cryptographic signature generated using the private key, such that the blockchain can verify the user has access to the corresponding digital directive. Once the user is verified, the user receives a link to access and edit the digital directive.

FIG. 8 illustrates an example method 800 for creating a digital token for a digital directive, in accordance with some embodiments of the present disclosure. In some embodiments, the method 800 is performed on the healthcare directive server 502 illustrated and described in reference to FIGS. 5 and 6. In alternative embodiments, the method 800 is performed at the patient user device. The method 800 includes the operations 802, 804, 806, and 808.

The operation 802 receives a completed digital directive and a public key from the patient user computing device. The public key is part of a private-public key pair, such that the public key can verify that a user possesses the proper private key to access the digital directive. In alternative embodiments, the operation 802 is performed at the patient user device. The operation 804 stores the completed digital directive in a digital directive data store. The operation 806 generates a digital token linking the digital directive to the public key. The operation 808 publishes the digital token to code to a blockchain. In some embodiments, the digital token is “minted” on the blockchain as part of the operation 808.

FIG. 9 illustrates an example method 900 for a delegate user to receive read-only access to the patients digital directive, in accordance with some embodiments of the present disclosure. The method 900 includes the operations 902, 904, 906, 908, 910, 912 and 914.

At the operation 902, the delegate computing device generates a private key and public key pair. In some embodiments, the public key corresponds to a public address for a digital wallet and the private key is used to sign transactions for the digital wallet. In some embodiments, the private key is further used to verify the user in order to access the digital directive.

At the operation 904, the delegate computing device provides the public key to the patient computing device. In some embodiments, the delegate computing device encodes a machine readable code (e.g., QR code) which is scannable to transfer the public key. In some embodiments, the public key is sent via an electronic message. At the operation 906, the patient computing device receives the public key from the delegate computing device.

At the operation 906, the patient computing device generates a transaction message to attach a read-only digital token to the public key from the delegate computing device. In some embodiments, the transaction message includes a cryptographic signature proving ownership of the digital directive by the patient using the patient's private key. In addition, to creating a cryptographic signature proving ownership of the digital directive the transaction message includes instructions to transfer a read-only digital token to the public key (e.g., wallet address) of the delegate user.

At the operation 908, the patient computing device sends the transaction message to the blockchain. In some embodiments, the patient computing device sends the transaction message to the blockchain via the healthcare directive server. In other embodiments, the patient computing device sends the transaction message directly to the blockchain.

At the operation 912, the blockchain verifies the transaction message. The blockchain will verify the transaction message, including verifying the cryptographic signature generated by the private key and verifying the public key for the patient computing device owns the associated digital directive.

At the operation 914, the blockchain records the transaction on the digital ledger. Once the transaction is recorded on the digital ledger the delegate computing device can access a read-only version of the digital directive using the private key to sign an access request certificate.

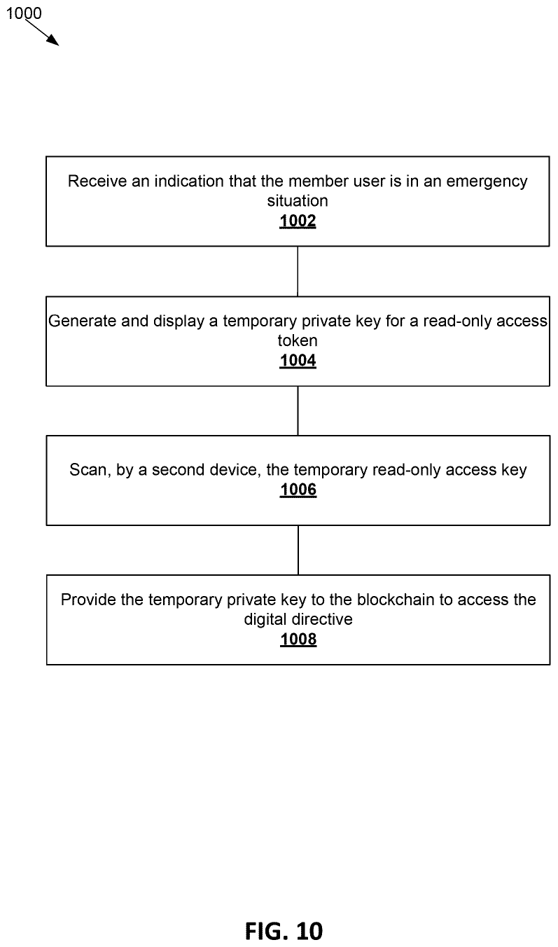

FIG. 10 illustrates an example method 1000 for receiving emergency access to a digital directive, in accordance with some embodiments of the present disclosure. The method 1000 includes the operations 1002, 1004, 1006, and 1008.

The operation 1002 receives, at a patient computing device, an indication that a patient is in an emergency situation. For example, the patient computing device may detect the user has lost pulse (e.g., via a connected heart rate monitor, such as a heart rate monitor on a smart watch) or the patient computing device may detect that the user has fallen and is not responding (e.g., via motion sensors and sending a notification to see if the user can respond). In some embodiments, an emergency professional has a special account which can send a message to a patient user computing device providing an indication that the user is in an emergency situation. In some embodiments, the indication is given by a manual input at the patient user computing device (e.g., as part of an emergency ID selection available when the patient user computing device is locked).

The operation 1004 generates and displays a temporary key for a read-only access token. In typical embodiments, the temporary key can only be used once. In other embodiments, the temporary key can be used until the patient user provides a selection to change or update the temporary key (e.g., by generating a new token with a new private-public key pair). In other embodiments, after the operation 1002 the patient user computing device generates the temporary read-only access token and publishes the read-only access token with a timer which limits the temporary key to only access the digital directive for a predetermined period of time. In some embodiments, the temporary private key for the read-only access token is displayed as part of a machine readable code (e.g., QR code) that the second computing device scans at the operation 1006. In alternative embodiments, the temporary private key is displayed in text which a second user can copy either automatically (e.g., by capturing an image via a camera and using computer vision to detect the code) or manually (e.g., by receiving user inputs entering the code).

The operation 1008 provides the temporary key to the blockchain in order to receive read only access the digital directive for the patient. In some embodiments, the second computing device renders a read-only digital directive on a display.

Although various embodiments are described herein, those of ordinary skill in the art will understand that many modifications may be made thereto within the scope of the present disclosure. Accordingly, it is not intended that the scope of the disclosure in any way be limited by the examples provided.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

What is claimed is:

1. A method for controlling access to a digital directive, the method comprising:
receiving the digital directive and a public key from a patient computing device;
storing the digital directive in a healthcare directive data store;
generating a digital token linking the digital directive to the public key; and
publishing the digital token to a blockchain, wherein the digital token, when combined with a cryptographic signature from a private key paired with the public key, allows access to the digital directive.

2. The method of claim 1, further comprising:
receiving, from the patient computing device, a cryptographically signed request to access the digital directive;
using the public key, determine whether the cryptographically signed request is signed using the private key; and
in response to determining that the cryptographically signed request is signed using the private key, providing read and write access to the digital directive to the patient computing device.

3. The method claim 2, wherein determining whether the cryptographically signed request is signed using the private key is performed at the blockchain.

4. The method of claim 2, further comprising, after providing read and write access to the digital directive to the patient computing device:
receiving an updated digital directive from the patient computing device;
storing the updated digital directive in the healthcare directive data store;
generating a second digital token linking the updated digital directive to the public key, wherein the second digital token is different from the digital token; and
publishing the second digital token to the blockchain, wherein the second digital token, when combined with the cryptographic signature from the private key paired with the public key, allows access to the updated digital directive.

5. The method of claim 1, further comprising:
receiving a delegate user public key;
generating a second digital token linking the delegate user public key with a read-only version of the digital directive; and
publishing the second digital token to the blockchain, wherein the second digital token, when combined with a second cryptographic signature from a delegate user private key paired with the delegate user public key, allows access to the read-only version of the digital directive.

6. The method of claim 5, wherein a delegate user computing device generates the delegate user public key and the delegate user private key and provides the delegate user public key to the patient computing device.

7. The method of claim 5, further comprising:
prior to generating the second digital token linking the delegate user public key with the read-only version of the digital directive, receiving a cryptographically signed transaction message from the patient computing device; and
sending the transaction message to the blockchain, wherein the blockchain is configured to verify the transaction message and record the transaction message on a digital ledger.

8. The method of claim 5, further comprising:
receiving, from a delegate user computing device, a second cryptographically signed request to access the digital directive;
using the delegate user public key, determine whether the second cryptographically signed request is signed using the delegate user private key; and
in response to determining that the cryptographically signed request is signed using the delegate user private key, provide read-only access to the digital directive to the delegate user computing device.

9. The method of claim 5, further comprising:
receiving a second delegate user public key;
generating a third digital token linking the second delegate user public key with a limited-view, read-only version of the digital directive; and
publishing the third digital token to the blockchain, wherein the third digital token, when combined with a third cryptographic signature from a second delegate user private key paired with the second delegate user public key, allows access to the limited-view, read-only version of the digital directive.

10. The method of claim 5, wherein a delegate user associated with the delegate user public key and the delegate user private key is a user with one of the following accounts:
(1) a physician account;
(2) a provider account;
(3) an emergency medical records account;
(4) a proxy user account;
(5) a family member account;
(6) a friend account;
(7) a lawyer user account;
(8) a payer user account,
(9) a hospice user account;
(10) a nursing home account;
(11) a hospice account; or
(12) an account for another decision maker associated with a patient user associated with the patient computing device.

11. The method of claim 1, wherein the healthcare directive data store is a decentralized database.

12. The method of claim 1, wherein the healthcare directive data store includes the blockchain.

13. The method of claim 1, further comprising:
anonymizing and encrypting user data associated with the digital token; and
providing the anonymized and encrypted user data to a researcher computing device in response to receiving a request signed by a research key.

14. The method of claim 1, wherein the public key corresponds to a public address for a digital wallet and the private key is useable to sign transactions for the digital wallet.

15. The method of claim 1, further comprising:
receiving, from a second computing device, an emergency access request signed using a temporary private key generated by the patient computing device;
verifying that the emergency access request is signed using the temporary private key; and
in response to verifying that the emergency access request is signed using the temporary private key, providing a read-only version of the digital directive to the second device.

16. A distributed system for controlling access to a digital directive comprising:
a patient computing device;
a blockchain
a healthcare directive server comprising:
a communication interface configured to establish digital communication with a healthcare directive data store, the patient computing device, and the blockchain;
at least one memory device; and
at least one processing device, the at least one memory device comprising instructions that, when executed by the at least one processing device, cause the healthcare directive server to:
receive a digital directive and a public key from the patient computing device;
store the digital directive in the healthcare directive data store;
generate a digital token linking the digital directive to the public key; and
publish the digital token to the blockchain, wherein the digital token, when combined with a cryptographic signature from a private key paired with the public key, allows access to the digital directive.

17. The system of claim 16, wherein the patient computing device comprises:
a second at least one memory device; and
a second at least one processing device, the second at least one memory device comprising second instructions that, when executed by the second at least one processing device, cause the patient computing device to:
generate the private key and the public key;
receive inputs for the digital directive;
upload the digital directive to the healthcare directive server with the public key; and
access the digital directive via the blockchain using the private key.

18. The system of claim 16,
further comprising a delegate user computing device;
wherein the instructions, when executed by the at least one processing device, further cause the healthcare directive server to:
receive a delegate user public key;
generate a second digital token linking the delegate user public key with a read-only version of the digital directive;
publish the second digital token to the blockchain, wherein the second digital token, when combined with a second cryptographic signature from a delegate user private key paired with the delegate user public key, allows access to the read-only version of the digital directive; and
provide the read-only version of the digital directive to the delegate user computing device after receiving a request signed by the delegate user private key.

19. A patient computing device comprising:
at least one memory device; and
at least one processing device, the at least one memory device comprising instructions that, when executed by the at least one processing device, cause the patient computing device to:
generate a private key and a public key corresponding to the private key;
receive inputs for a digital directive;
provide the digital directive and the public key to a healthcare directive server to store the digital directive in a healthcare directive data store;
generate a digital token linking the digital directive to the public key; and
publish the digital token to a blockchain, wherein the digital token enables access to the digital directive when using the private key.

20. The patient computing device of claim 19,
wherein the patient computing device is communicatively coupled with a sensor of a smart device; and
wherein the instructions, when executed by the at least one processing device, cause the patient computing device to:
receive, from the smart device, data collected by the sensor indicating that a patient associated with the patient computing device is in critical condition; and
generate a temporary private key for accessing a read-only version of the digital directive.
